(19) United States
(12) Patent Application Publication
(10) Pub. No.: US 2004/0064149 A1
(43) Pub. Date: Apr. 1, 2004

(54) Probe holder to facilitate fibre optic examination of tissue surfaces

(76) Inventors: Frederick E. Doern, Hazelridge (CA); Gary J. Young, Winnipeg (CA)

Correspondence Address: Ade & Company, 1700-360 Main Street, Winnipeg, MB, R3C 3Z3, CA

(21) Appl. No.: 10/667,416

(22) Filed: Sep. 23, 2003

Related U.S. Application Data
(60) Provisional application No. 60/412,552, filed on Sep. 23, 2002.

(51) Int. Cl. A61B 19/00

(52) U.S. Cl. 606/130

(57) ABSTRACT

A probe holder for holding a fibre optic probe is described. The probe holder comprises a probe engagement member and supports extending outwardly from the probe engagement member.

This application claims priority under 35 USC § 119(e) to Provisional Patent Application Serial No. 60/412,552 filed on Sep. 23, 2002.

### FIELD OF THE INVENTION

The present invention relates generally to the field of medical devices. More specifically, the present invention relates to a fibre optic probe holder.

### BACKGROUND OF THE INVENTION

The prior art describes several devices arranged for spectrophotometric examination of tissue portions using fibre optic probes. Typically, these devices use a variety of light sources, for example, visible light, infra-red light and near infrared light to examine analytes within the tissue portion.

For example, fibre optic devices have been used to monitor tissue oxygenation and perfusion. In some instances, these devices are used to monitor trends in oxygenation and blood flow of tissue portions wherein blood flow from the tissue portion to the main body has been compromised, for example, as a result of injury, burn damage, freezing or a transplant or graft.

The prior art teaches mounting the fibre optic probe directly onto the tissue portion to be examined. As will be appreciated by one of skill in the art, particularly in instances where the tissue portion has been damaged, mounting the probe directly onto the tissue portion may further damage the tissue portion, causing pain and discomfort of the patient and slowing healing.

Clearly, a device which is arranged to support a fibre optic probe at a fixed distance away from a tissue portion so that the tissue portion is not further compromised by contact with the probe is needed.

### SUMMARY OF THE INVENTION

According to a first aspect of the invention, there is provided a probe holder comprising:

a probe engagement member comprising:

an opening arranged to accept a probe head therein; and

sidewalls extending upwardly from the opening, said sidewalls for supporting the probe head; and

supports extending outwardly from the probe engagement member.

According to a second aspect of the invention, there is provided a method of using a fibre optic probe on a patient in need of such, comprising:

providing a patient having damaged tissue surrounded by substantially healthy tissue;

providing a probe holder comprising:

a probe engagement member comprising:

an opening arranged to accept a probe head therein; and

sidewalls extending upwardly from the opening, said sidewalls for supporting the probe head; and

supports extending outwardly from the probe engagement member,

mounting the probe holder onto the patient such that the probe engagement member is opposite the tissue to be examined and the supports rest substantially on the surrounding tissue; and

fitting a fibre optic probe into the probe holder.

### BRIEF DESCRIPTION OF THE DRAWINGS

FIG. 1 shows one embodiment of the probe holder in perspective.

FIG. 2 is a top view of a probe holder.

FIG. 3 shows a side view of the probe engagement member.

FIG. 4 shows a side view of an alternate arrangement of the probe engagement member.

FIG. 5 shows a side view of an alternate arrangement of the probe engagement member.

FIG. 6 shows a perspective view of an alternate arrangement of the probe engagement member.

FIG. 7 shows a top plan view of an alternative embodiment of the probe holder.

FIG. 8 shows a side view of an alternate arrangement of the probe engagement member.

FIG. 9 shows a perspective view of an alternate arrangement of the probe engagement member.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As is known to one of skill in the art, the basic components of a fibre optic probe device includes those elements common to a spectrophotometric device. These include but are by no means limited to an irradiation (light) source (broadband tungsten or metal halide light source, light emitting diode, “Nernst glow-bar”, deuterium lamp, or other suitable source for wavelength-enhanced ultraviolet and/or/through infrared emission); a set of source optics (lens or fibre optic elements with or without a monochromator if a monochromatic source is required); a set of detection optics (fibre optic or lens elements coupled with a dispersion element such as a prism or diffraction grating); an energy specific detector such as a charge coupled device, bolometer, or the like; a power source(s) to supply appropriate power requirements for the various components; a display mechanism to record and display the spectrum of the material under examination; and a microcomputer or microcontroller to control servo-mechanisms for specimen irradiation, data collection, data display, and operator interaction with the device.

Referring to the drawings, the probe holder 1 comprises a substantially vertical probe engagement member 10 and supports 12 extending horizontally outwards therefrom. As a result of this arrangement, when the probe holder 1 is mounted onto a tissue to be examined, the contact pressure against the skin is spread out over the length of the probe holder, thereby preventing discomfort and blanching in the area of interest. As will be appreciated by one of skill in the art, this is particularly important when the tissue to be examined has been damaged or compromised, for example, burned or frozen or has been recently grafted and is highly contact-sensitive. As discussed below, during examination, a probe head is inserted into the probe engagement member 10, thereby securing the probe head at a specific distance from the tissue to be examined and orientation relative to the tissue. It is also of note that the probe head may be removed from the probe holder 1 if necessary and reinserted at a later time into the probe engagement member 10 with the probe head being at the same distance and orientation relative to the tissue portion of interest as previously.

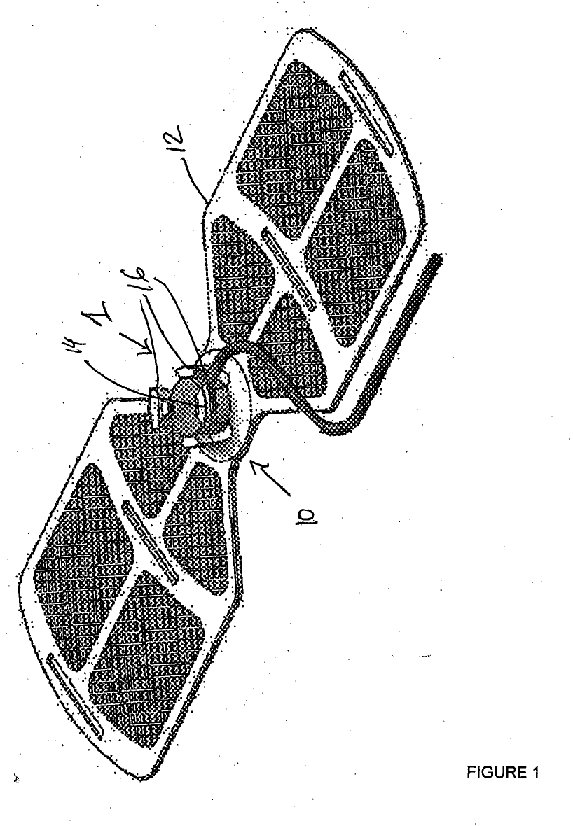

As discussed below, in some embodiments, the probe holder 1 is arranged such that the supports 12 are connected to the probe engagement member 10 such that the probe engagement member 10 is supported above the plane of the supports 12. As will be appreciated by one of skill in the art, in these arrangements, the probe holder 1 is arranged such that the probe engagement member is supported above the tissue to be examined. Thus, as can be seen in FIG. 1, the supports 12 extend outwardly from the probe engagement member 10 and support the probe engagement member 10 above the plane of the supports 12. As a result of this arrangement, as discussed below, when the probe holder is mounted onto a patient, the probe engagement member 10 does not contact the tissue portion being examined by the fibre optic probe. Furthermore, the fibre optic probe or probe head is held at a fixed position (both orientation and distance) relative to the tissue portion, as discussed above.

As will be appreciated by one of skill in the art, the supports 12 may be composed of a plurality of suitable materials and more preferably, may be composed of a material that is non-allergenic and non-irritating to the skin. Furthermore, the supports 12 are sufficiently flexible as to conform to body curvatures. As a result of this arrangement, the supports 12 may be secured, for example, by medical tape, straps or the like, to the skin of a patient without causing additional irritation or discomfort.

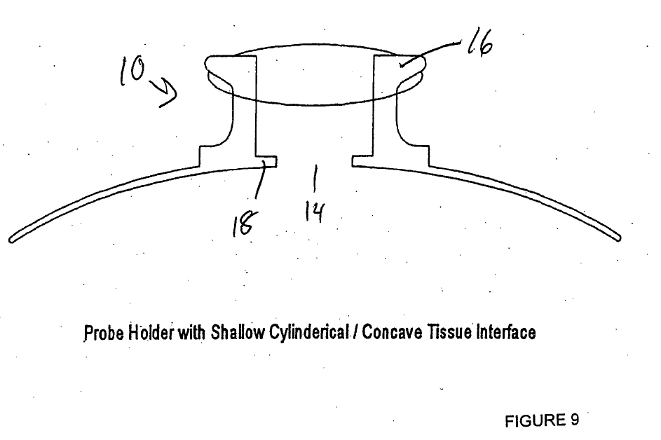

The probe engagement member 12 comprises an opening 14 and sidewalls 16 extending upwardly therefrom, as shown in FIGS. 3, 4, 5, 6, 8 and 9.

As can be seen in FIGS. 3, 4, 5 and 9, in some embodiments, the probe engagement member also includes a lip 18 extending around the outer perimeter or circumference of the opening 14 (depending on the specific shape of the opening 14). In this arrangement, the base of the probe head rests on the lip 18 such that the probe head projects through the opening 14. It is of note that the lip 18 thereby defines a distance “d” between the tissue surface and the active surface of the probe head. As will be apparent to one of skill in the art, the thickness of the lip 18 may be varied in different embodiments of the probe holder 1 so that the probe head is supported away from the tissue portion at variable distances, as discussed below.

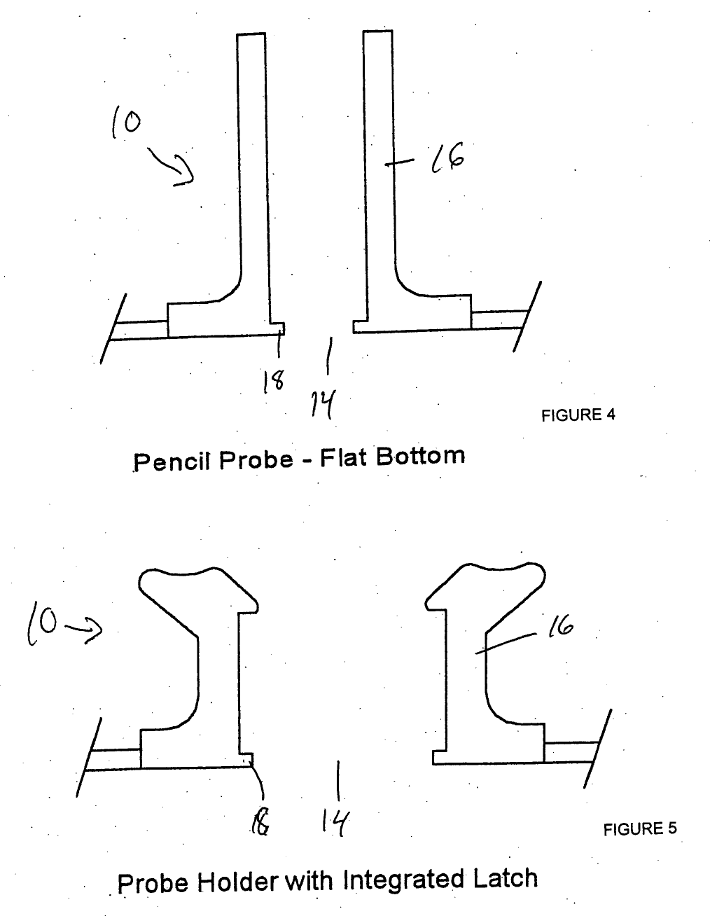

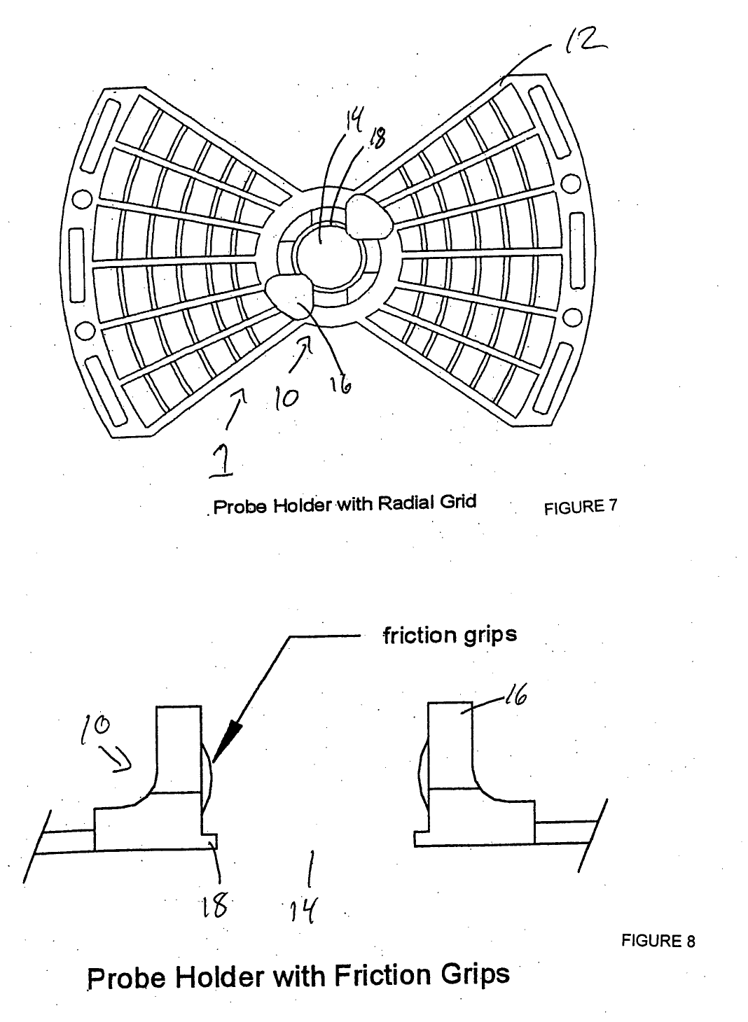

As can be seen in the accompanying Figures, the sidewalls 16 effectively form a socket for supporting and/or engaging the fibre optic probe or probe head. As will be appreciated by one of skill in the art, a wide variety of possible probe engagement member arrangements are within the scope of the invention. For example, the sidewalls 16 may be tapered to facilitate insertion of the probe head (FIG. 3). Alternatively, the sidewalls 16 may be elongated, thereby forming heightened walls to accommodate “pencil-like” or “wand-like” fibre optic probe designs (FIG. 4). In the embodiment shown in FIG. 5, the sidewalls 16 include integrated latches 18 for engaging the probe head. In the embodiment shown in FIG. 8, the sidewalls 16 include friction grips 20 for engaging the probe head.

Thus, in some embodiments, the probe engagement member, by use of a suitable choice of materials, holds the probe head by for example, friction and/or positive locking. In the friction method, the body of the probe holder part grips the periphery of the probe head and is intended, in part, to provide the lowest profile holding arrangement. In the positive locking method, the probe head is held in the probe holder part by clasps or clips, that can be part of the probe holder, a portion of which slide over the opposite end of the probe head. The positive locking method is intended, in part, to allow removal of the probe head from the probe holder while the probe holder remains fixed to the patient and conversely, allows the probe head to be reinstalled in the probe holder in exactly the same position as it was before removal.

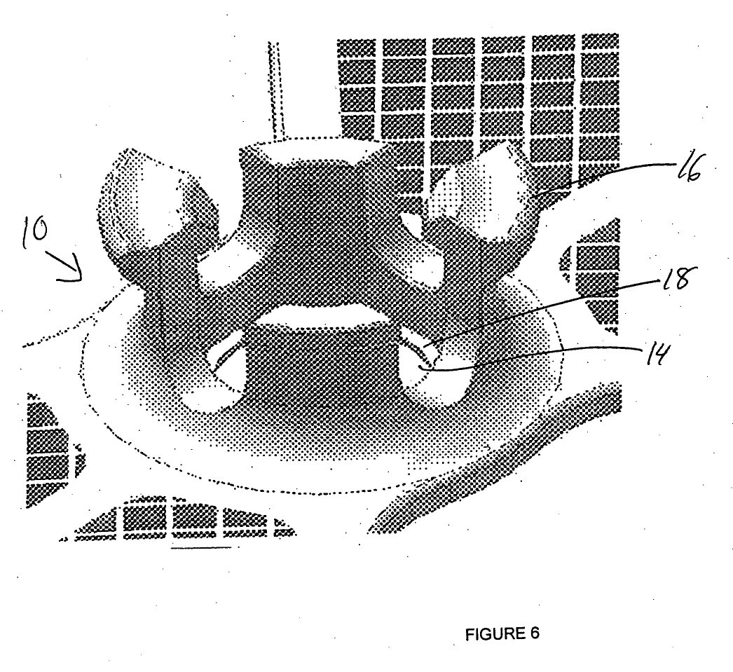

The clamps on the positive locking method are designed to allow the use of an external device to apply additional force to the clamping arms, for example, an elastic band as shown in FIG. 6.

The clamps on the positive locking method are designed to facilitate the disengagement by hand for easy removal of the probe head.

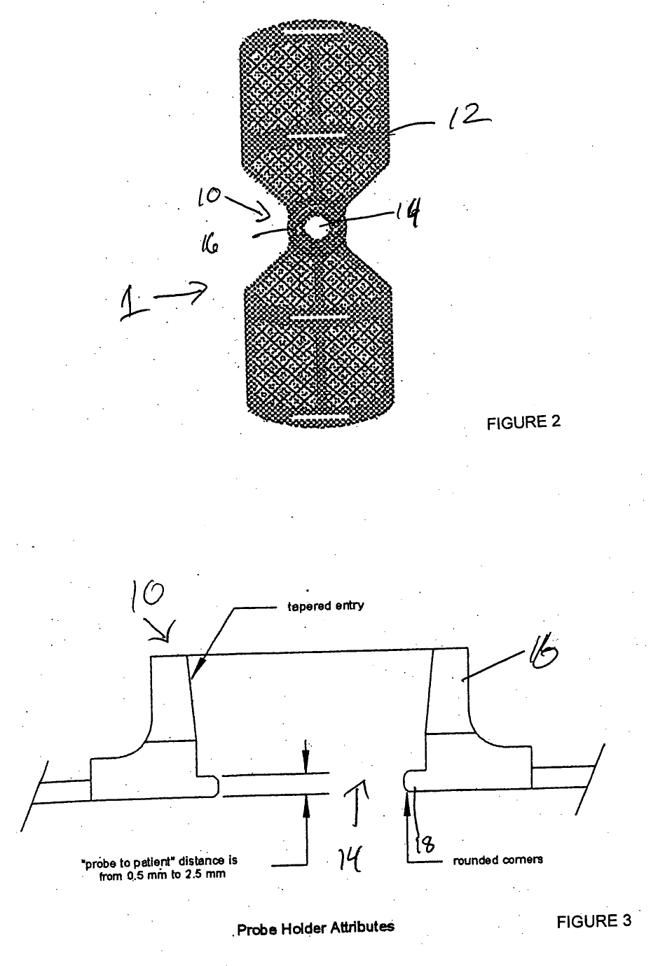

It is of note that in FIGS. 1, 2 and 7, there are two supports 12 positioned on opposite sides of the probe engagement member 10. As will be appreciated by one of skill in the art, this is not a necessary feature of the invention and more than two supports may be used in some embodiments. Additionally, in some embodiments, the supports 12 may be off-set relative to one other, that is, not directly opposite each other.

It is of note that the supports 12 may be arranged to be easily cut or trimmed for customizing the length of the probe holder 1 so as to facilitate meeting immediate attachment requirements. In other embodiments, the supports 12 may include a plurality of breakpoints for facilitating adjusting the length of the probe holder 1 and to facilitate contact points to attach the probe holder to healthy tissue sites. However, the probe holder 1 is arranged to be dimensionally stable under reasonable tension.

As will be appreciated by one of skill in the art, the actual dimensions of the probe holder 1 may vary greatly, depending on the intended use, and are within the scope of the invention.

The supports 12 may be of a material or physical construction which permits gas flow, for example, air or oxygen, to the tissue portion. Thus, the supports 12 do not compromise oxygen transport to the tissue. Furthermore, as can be seen in FIGS. 1, 2 and 7, in some embodiments, the supports 12 of the probe holder 1 have sufficient open space to permit visual assessment of the flap or tissue area while the probe holder is still in place.

It is of note that the probe holder 1 may be composed of any non-toxic, non-allergenic polymer that is readily molded and can be easily sterilized through common medical sterilization procedures. These include, for example, although my no means limited to polyethylene, polypropylene, and combinations thereof or these materials in combination with other suitable materials.

It is of note that the supports 12 are arranged to spread any contact pressure between the tissue and probe assembly over a much larger surface area and as a consequence do not induce any “blanching effect” or undue pressure on the tissue portion that might induce erroneous readings for tissue viability.

It is further of note that the probe holder may be manufactured for different probe diameters and or probe geometries.

Thus, the probe holder 1 is designed to facilitate continuous monitoring of tissue using spectrophotometric methods involving fibre optic probes for the delivery of light and detection of characteristic spectral signatures.

The probe holder acts as an optical positioning device; as it holds the fibre optic probe in position normal to the tissue surface and at a pre-defined distance from the surface. This latter point becomes more important when we consider the numerical aperture of the fibres and relative distance between source and detector fibres. The diameter of the irradiation spot(s) will vary as a function of the numerical aperture of the fibre and the distance (d) between the planar end of the fibre optic probe and the tissue surface.

The probe holder 1 may be used in conjunction with a sterile optical sheath to provide an effective biological barrier between the probe surface and the tissue being examined. This optical sheath need only encase the probe and not the probe holder, which in turn is sterilized prior to use.

It is further of note that the probe holder 1 is in some embodiments arranged for single use (disposable). Furthermore, the probe holder 1 is compatible with common sterilization methods and with common medical device packaging techniques.

In some embodiments, the supports 12 are designed for common attachments in at least one location along the length or on the width. In yet other embodiments, common attachments may be built into the manufacture of the probe holder for specific applications.

As discussed above, the probe engagement member fixes the probe head at a fixed distance from the skin surface. In some embodiments, this probe head may be supported at the tissue surface. In yet other embodiments, the probe head may be 0.1 mm to 0.5 mm away from the tissue to be examined. In yet other embodiments, the probe engagement member 10 may be manufactured for other fixed distances, for example, 1 mm, 1.5 mm, 2.0 mm or 2.5 mm. As discussed above, the probe holder 1 may hold the probe head at a fixed distance away from the tissue surface when in use either by having a lip 18 of variable thickness or supporting the probe engagement member 10 above the plane of the supports 12 or a combination thereof.

It is of note that the probe head may be encased in a sheath for maintaining sterility. As will be appreciated by one of skill in the art, this arrangement ensures a biological barrier between the tissue and the probe head. This will in turn reduce the wear and tear on the fibre optic probe which would therefore not need to be subjected to formal sterilization procedures. It is of note that in some embodiments the probe holder 1 may be arranged such that the probe engagement member 10 includes a sterile sheath extending across the opening 14.

In some embodiments, the probe holder part may have indentations or cut-outs for fixing the Probe Head and corresponding optical cable in a specific orientation.

The probe engagement member may be manufactured of opaque material to reduce stray light contamination when used in high levels of ambient lighting.

For use, there is provided a patient having a damaged or compromised tissue portion surrounded by tissue that is undamaged, healthier or less at risk. The probe holder 1 is positioned onto the patient such that the supports 12 rest substantially on undamaged or less at risk tissue and the opening 14 on the probe engagement member is opposite at least a portion of the damaged tissue. As discussed above, the supports 12 of the probe holder 1 may be trimmed or cut such that the length of the probe holder 1 is suitable and relatively comfortable for the patient. As discussed above, the probe holder 1 is then secured to the patient using for example straps or medical tape, although any suitable method of securing the probe holder 1 to the patient may be used. As a result of this arrangement, blanching of the tissue to be examined is minimized. The probe head is then fitted into the probe engagement member 10 such that the tip of the probe head extends through the sidewalls 16 and into the opening 14. As discussed above, the sidewalls 16 engage and support the body of the probe head, thereby securing the probe head at a fixed distance from the damaged tissue portion. As discussed above, the probe holder 1 may include for example clips or a similar structure for securing the fiber optic cable thereto.

As discussed above, in some embodiments, the probe head rests on the lip 18 extending around the opening 14, thereby supporting the probe head at a fixed distance away from and above the tissue. In other embodiments, the probe holder 1 is arranged such that the supports 12 rest on the undamaged tissue while the probe head or probe engagement member 10 is supported above the plane of the supports 12 and therefore does not contact the damaged tissue portion. It is of note that in yet other embodiments, these two features may be combined in a single probe holder 1.

In use, the probe head analyses the damaged tissue portion. As noted above, if necessary, the probe head may be disconnected from the probe holder 1 and reconnected at a later time, with the probe head remaining at the previous distance from the damaged tissue portion.

As discussed above, the supports 12 of the probe holder 1 are composed of non-allergenic and non-irritating material and the probe holder 1 is flexible enough to conform with the contours of the patient's body.

While the preferred embodiments of the invention have been described above, it will be recognized and understood that various modifications may be made therein, and the appended claims are intended to cover all such modifications which may fall within the spirit and scope of the invention.

1. A probe holder comprising:
a probe engagement member comprising:
an opening arranged to accept a probe head therein; and
sidewalls extending upwardly from the opening, said sidewalls for supporting the probe head; and
supports extending outwardly from the probe engagement member.

2. The probe holder according to claim 1 wherein the probe engagement member is connected to the supports such that the probe engagement member is above a plane of the supports.

3. The probe holder according to claim 1 including a lip extending around the opening for supporting the probe head thereon.

4. The probe holder according to claim 1 wherein the supports are composed of a non-irritating material.

5. The probe holder according to claim 1 wherein the supports are composed of a non-allergenic material.

6. The probe holder according to claim 1 wherein the sidewalls are tapered towards the opening.

7. The probe holder according to claim 1 wherein the sidewalls include friction grips for securing the probe head.

8. The probe holder according to claim 1 wherein the supports are arranged to be cut or broken.

9. The probe holder according to claim 1 wherein the supports are substantially air permeable.

10. The probe holder according to claim 1 wherein the probe holder is composed of a substantially opaque material.

11. A method of using a fibre optic probe on a patient in need of such, comprising:
providing a patient having damaged tissue surrounded by substantially healthy tissue;
providing a probe holder comprising:
a probe engagement member comprising:
an opening arranged to accept a probe head therein; and
sidewalls extending upwardly from the opening, said sidewalls for supporting the probe head; and
supports extending outwardly from the probe engagement member,
mounting the probe holder onto the patient such that the probe engagement member is opposite the damaged tissue and the supports rest substantially on the substantially healthy tissue; and
fitting a fibre optic probe into the probe holder.

12. The method according to claim 11 wherein the fibre optic probe emits through the opening onto the tissue portion.

13. The method according to claim 11 wherein the probe engagement member is connected to the supports such that the probe head is above a plane of the supports.

14. The method according to claim 11 wherein the probe holder includes a lip surrounding the opening for supporting the probe head thereon.

15. The method according to claim 11 wherein the supports are composed of a non-irritating material.

16. The method according to claim 11 wherein the supports are composed of a non-allergenic material.

17. The method according to claim 11 wherein the sidewalls are tapered towards the opening.

18. The method according to claim 11 wherein the sidewalls include friction grips for securing the probe head.

19. The method according to claim 11 wherein the supports are arranged to be cut or broken.

20. The method according to claim 11 wherein the supports are substantially air permeable.

21. The method according to claim 11 wherein the probe holder is composed of a substantially opaque material.
